(19) United States
(12) Patent Application Publication
(10) Pub. No.: US 2025/0224333 A1
(43) Pub. Date: Jul. 10, 2025

(54) THERMAL PROPERTY MEASUREMENT METHOD FOR 3D THERMAL IMAGING

(71) Applicant: Taiwan Semiconductor Manufacturing Company, Ltd., Hsinchu (TW)

(72) Inventors: James June Fan Hsu, New Taipei City (TW); Che Chi Shih, Taoyuan City (TW); Wei-Yen Woon, Taoyuan City (TW); Ku-Feng Yang, Hsinchu County (TW); Han-Yu Lin, Nantou County (TW); Kuan-Kan HU, Hsinchu (TW); Chun-Yu Liu, New Taipei City (TW); Szuya LIAO, Hsinchu (TW)

(73) Assignee: Taiwan Semiconductor Manufacturing Company, Ltd., Hsinchu (TW)

(21) Appl. No.: 18/404,751

(22) Filed: Jan. 4, 2024

(51) Int. Cl. G01N 21/55; G01N 1/44

(52) U.S. Cl. CPC G01N 21/55; G01N 1/44

(57) ABSTRACT

High resolution 3D thermal imaging can be obtained by using enhanced non-destructive heat transducer designs. A thermal property measurement method includes providing a sample for thermal property measurement, and bonding a transducer layer on the sample through a temporary bonding layer. Thermal measurement processes are performed along the X-Y, X-Z and Y-Z planes of the sample, wherein the X-Y plane is parallel to a top surface of the sample, and the X-Z plane and Y-Z plane are perpendicular to the top surface of the sample. Each thermal measurement processes include heating a designated region of the sample covered with the transducer layer using a pump laser, and using a probe laser for generating a reflectance signal of the sample, and determining a thermal conductivity in the designated region of the sample from the reflectance signal. Furthermore, the transducer layer is removed along with the temporary bonding layer from the sample.

### BACKGROUND

In recent years, non-contact laser pump-probe techniques such as steady-state thermoreflectance (SSTR) based optical pump-probe techniques were introduced for directly measuring thermal properties (thermal conductivity K) of different material films and interfaces. SSTR metrology can measure thermal conductivity of materials or structures by measuring thermal reflectance change (ΔR). SSTR uses a continuous wave (CW) pump laser to optically excite thermal reflectance changes of the other probe laser.

### BRIEF DESCRIPTION OF THE DRAWINGS

Aspects of the disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1A to FIG. 1B are schematic sectional and three-dimensional views of various steps in a thermal property measurement method in accordance with some embodiments of the present disclosure.

FIG. 2 to FIG. 8 are schematic sectional views of various steps in performing thermal measurement processes along a X-Y plane in the thermal property measurement method in accordance with various embodiments of the present disclosure.

FIG. 9 to FIG. 10C are schematic sectional views of various steps in performing thermal measurement processes along a X-Z plane or a Y-Z plane in the thermal property measurement method in accordance with various embodiments of the present disclosure.

FIG. 11A to FIG. 13B are schematic sectional and three-dimensional views of various steps in performing thermal measurement processes along a X-Y plane in the thermal property measurement method in accordance with some other embodiments of the present disclosure.

FIG. 14A to FIG. 15B are schematic sectional views of various steps in performing thermal measurement processes along a X-Z plane or a Y-Z plane in the thermal property measurement method in accordance with some other embodiments of the present disclosure.

### DETAILED DESCRIPTION

The following disclosure provides many different embodiments, or examples, for implementing different features of the provided subject matter. Specific examples of components, values, operations, materials, arrangements, or the like, are described below to simplify the disclosure. These are, of course, merely examples and are not intended to be limiting. Other components, values, operations, materials, arrangements, or the like, are contemplated. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

Non-contact laser pump-probe techniques such as steady-state thermoreflectance (SSTR), can be used for directly measuring thermal properties (thermal conductivity K) of different material films and interfaces. However, traditional SSTR metrology only measures a single spot per time, and the measurement of thermal conductivity is performed non-directionally by quantifying the overall heat dissipation capacity for materials/structures. In other words, traditional SSTR offers a harmonic average thermal conductivity k in all directions, but the directional thermal conductivity of heat transport design structures, such as thermal vias and heat spreaders cannot be evaluated. Furthermore, the measured signal (normalized change in reflectivity ΔR/R) of traditional SSTR is also very small (approximately 10E-3).

In some embodiments of the present disclosure, the SSTR technique is extended for three-dimensional (3D) mapping of thermal properties, which will meet the demands in in characterizing 3D heat dissipation in developing transistors and chip bonding structures, or directional heat transport design structures in the back-end-of Line (BEOL) of fabrication. The SSTR technique can be tuned to directly measure the in-plane and out-of-plane thermal conductivity, with measurement performed at the desired location and spatial resolution.

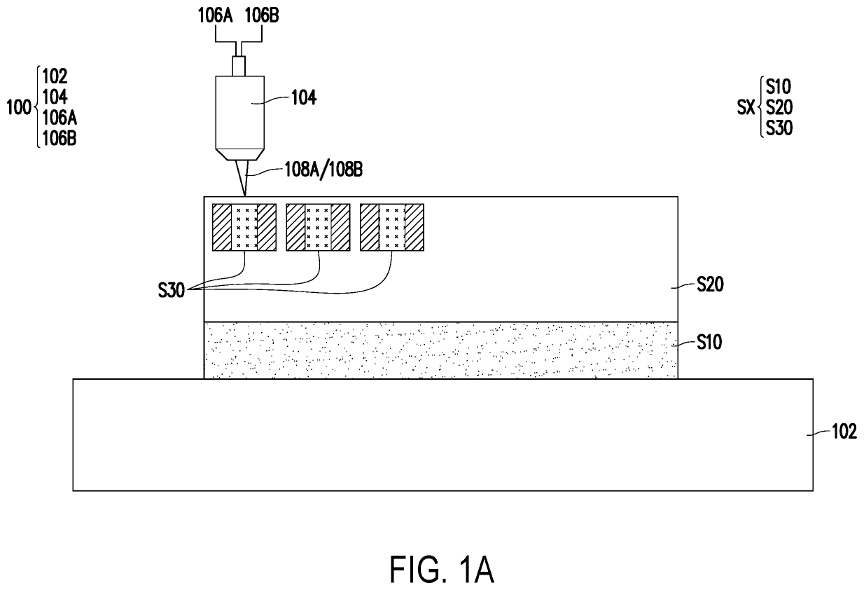

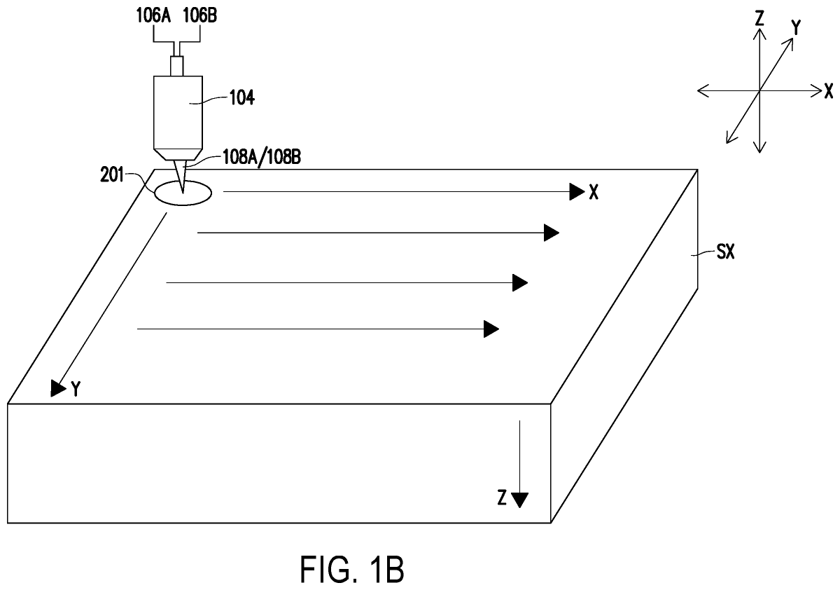

FIG. 1A to FIG. 1B are schematic sectional and three-dimensional views of various steps in a thermal property measurement method in accordance with some embodiments of the present disclosure. Referring to FIG. 1A to FIG. 1B, in some embodiments, a thermal property measurement method (or method of measuring thermal conductivity) of the present disclosure includes using a thermal measurement system 100 for measuring thermal properties. As illustrated in FIG. 1A, in some embodiments, the thermal measurement system 100 includes at least a sample stage 102 (or sample holder), an objective lens 104, a pump laser 106A and a probe laser 106B. The sample stage 102 is used for holding a sample SX for thermal measurement. The objective lens 104 is used for focusing the emitted laser onto the sample SX. The pump laser 106A (continuous wave pump laser) is configured to emit a first laser beam 108A that is sent through the objective lens 104 to focus onto the sample SX, and for heating the sample SX. The probe laser 106B is configured to emit a second laser beam 108B that is sent through the objective lens 104 to focus onto the sample SX, and for detecting a thermal reflectance change of the sample SX.

In some embodiments, the first laser beam 108A emitted by the pump laser 106A provides a radial heat flux to the sample SX for heating a designated region or designated spot 201 (see FIG. 1B) of the sample SX. Furthermore, the second laser beam 108B emitted by the probe laser 106B is focused on the sample SX to generate a reflected probe beam having a reflectance signal. In some embodiments, the thermal reflectance change over time of the sample SX is determined by measuring a magnitude of the reflectance signal. In addition, a temperature change and a thermal conductivity of the sample at the designated spot 201 can be calculated from the thermal reflectance change. In other words, by measuring the reflectance signal of the second laser beam 108B (probe beam) reflected from the sample SX, the measured data can be fitted to a thermal model to determine the thermal conductivity.

As illustrated in FIG. 1B, in the thermal property measurement method of the present disclosure, a single thermal measurement process is performed by heating a designated spot 201 (a first designated spot) of the sample SX using the pump laser 106A, and using the probe laser 106B for generating a reflectance signal from the designated spot 201 of the sample SX. In the exemplary embodiment, the thermal measurement process is repeated to obtain the temperature change and the thermal conductivity of the sample SX at various spots (a second designated spot, a third designated spot, etc.) along a X-direction, along a Y-direction and along a Z-direction of the sample SX, wherein the Y-direction is perpendicular to the X-direction, and the Z-direction is perpendicular to the X-direction and the Y-direction. In other words, the scanning of the pump laser 106A and the probe laser 106B are performed to directly measure an in-plane (the X-Y plane) and out-of-plane (the X-Z plane and Y-Z plane) thermal properties of the sample SX. The in-plane (the X-Y plane) thermal properties is measured at the plane that is parallel to a top surface of the sample SX, whereby the out-of-plane (the X-Z plane and Y-Z plane) thermal properties is measured at the planes that are perpendicular to the top surface of the sample SX.

In the exemplary embodiment, by performing the in-plane (the X-Y plane) and out-of-plane (the X-Z plane and Y-Z plane) thermal measurements, a three-dimensional thermal image of the sample SX can be generated by mapping the temperature change and the thermal conductivity of the sample SX obtained at the various spots along the X-direction, the Y-direction and the Z-direction.

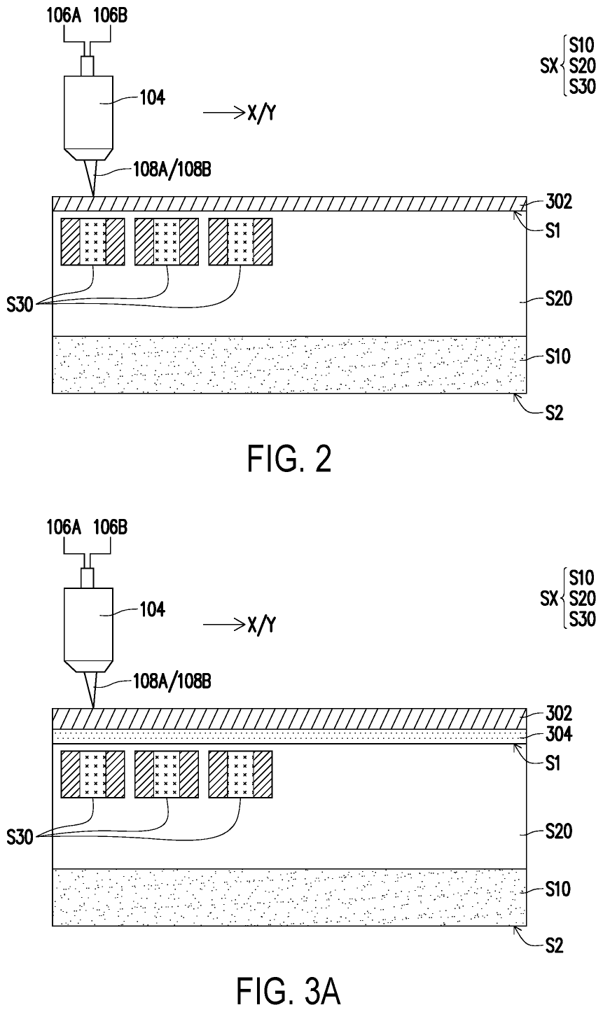

In some embodiments, the measured sample SX is a semiconductor device including a semiconductor substrate S10, dielectric layers S20 disposed on the semiconductor substrate S10, and transistors S30 embedded in the dielectric layers S20. By using the thermal property measurement method of the present disclosure for 3D mapping, it is possible to monitor steady state thermal responses of semiconductor devices in a specific direction and monitor locations mimicking where electrical devices (e.g. transistors) operate and dissipate heat. For example, the heat dissipation characteristics of the transistors S30 can be evaluated through 3D mapping of the thermal properties. Although the sample SX in the illustrated embodiment is directed to a semiconductor device, the disclosure is no limited thereto. In some alternative embodiments, the thermal property measurement method of the present disclosure can also be used for measuring the thermal properties of different material films and interfaces. The details of performing the thermal measurement processes along the X-Y plane or along the X-Z plane and Y-Z plane will be described in more detail by referring to FIG. 2 to FIG. 15B. In the embodiments illustrated in FIG. 2 to FIG. 15B, the same reference numerals are used for referring to the same or like parts, and their detailed description may not be repeated therein.

FIG. 2 to FIG. 8 are schematic sectional views of various steps in performing thermal measurement processes along a X-Y plane in the thermal property measurement method in accordance with various embodiments of the present disclosure. Referring to FIG. 2, in some embodiments, after providing the sample SX on the sample stage 102, and prior to performing the thermal measurement process along the X-direction and the Y-direction, a transducer layer 302 is provided onto a top surface S1 of the sample SX. Although the transducer layer 302 is shown to be placed on the top surface S1 of the sample SX, the disclosure is not limited thereto. In some alternative embodiments, the transducer layer 302 is placed on a bottom surface S2 of the sample SX based on measurement requirements. In some embodiments, the transducer layer 302 comprises metal thin films having a thickness of 20 nm to 80 nm. In certain embodiments, the transducer layer 302 is for example, an aluminum (Al) layer, a gold (Au) layer, a copper (Cu) layer, a plutonium (Pu) layer, or a bi-layer consisting of aluminum and gold. In the exemplary embodiment, the transducer layer 302 assist in the heating of the sample SX using the pump laser 106A, and the reflectance of the transducer layer 302 changes with the temperature rise, thus allowing the detection of the thermal response by monitoring the reflectance change. By tuning the materials of the transducer layer 302 a heat absorption of the transducer layer 302 may be improved. For example, in some embodiments, a heat absorption of using a bi-layer consisting of aluminum and gold as the transducer layer 302 is two to three times higher than a heat absorption of using a bulk aluminum layer as the transducer layer 302.

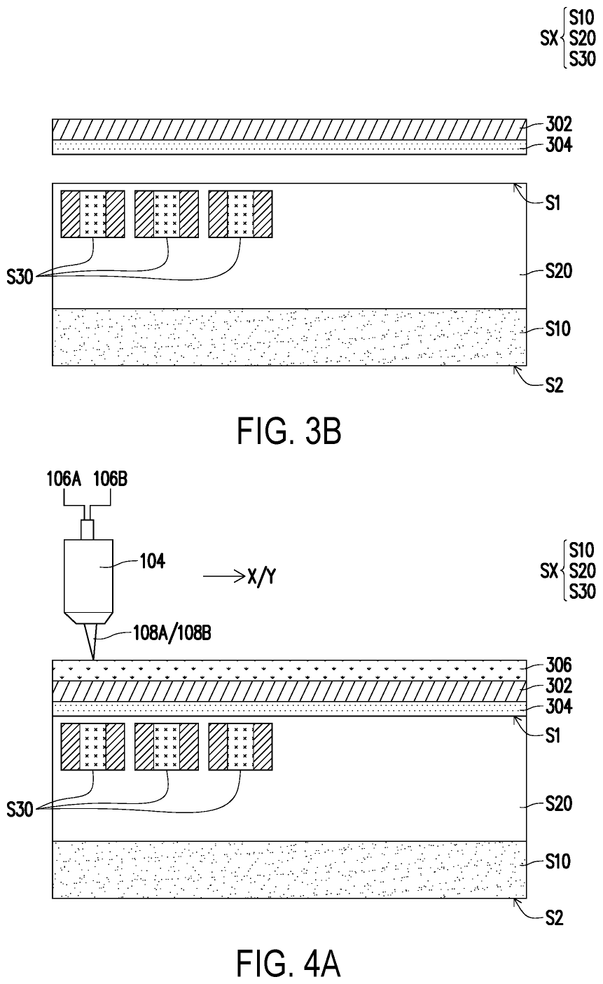

Referring to FIG. 3A to FIG. 3B, in some embodiments, the transducer layer 302 is bonded to the top surface S1 of the sample SX through a temporary bonding layer 304. In other words, the temporary bonding layer 304 is sandwiched in between the top surface S1 of the sample SX and the transducer layer 302. The temporary bonding layer 304 includes a thermally conductive material. For example, the temporary bonding layer 304 is selected from the group consisting of germanium (Ge), silicon germanium (Si₁₋ₓGeₓ; where x=0.7˜1), silicon carbide (SiC), phosphorus-doped silicon (Si:P), boron-doped silicon (Si:B), boron-doped silicon germanium (SiGe:B), phosphorus-doped silicon germanium (SiGe:P), yttrium oxide (Y₂O₃), cerium oxide (CeO₂), boron nitride (BN), gallium phosphide (GaP), and titanium nitride (TiN). In some embodiments, the temporary bonding layer 304 may be a single crystal material, a polycrystalline material, or an amorphous material, and may have a cubic, hexagonal, tetragonal, orthorhombic, monoclinic, triclinic crystal system. In some embodiments, the temporary bonding layer 304 may be grown on the transducer layer 302 by physical vapor deposition (PVD), plasma enhanced atomic layer deposition (PEALD), thermal atomic layer deposition (thermal ALD), microwave-plasma chemical vapor deposition (MWCVD), plasma-enhanced chemical vapor deposition (PECVD), thermal chemical vapor deposition (thermal CVD) and hybrid physical-chemical vapor deposition (HPCVD) at temperatures of 100° C. to 1400° C. In the exemplary embodiment, by using the above materials as the temporary bonding layer 304, an absorption and reflectance of the transducer layer 302 on the sample SX will not be affected.

In the exemplary embodiment, since the transducer layer 302 is bonded to the top surface S1 of the sample SX through a temporary bonding layer 304 during the thermal measurement process shown in FIG. 3A, and the temporary bonding layer 304 along with the transducer layer 302 are removed (e.g. through light or heat) after measurement as shown in FIG. 3B, the damage to the sample SX can be minimized. In other words, the thermal property measurement method of the present disclosure is a non-destructive method, and when the sample SX is a semiconductor device, further fabrication processes may be continued on the surfaces of the sample SX after the temporary bonding layer 304 is removed.

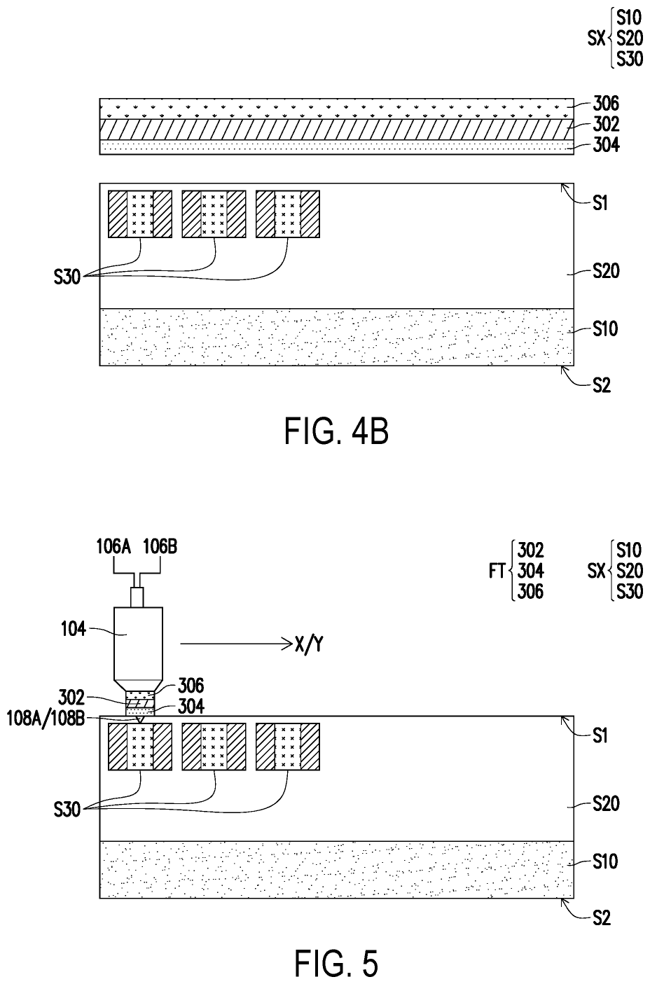

Referring to FIG. 4A, in some embodiments, prior to performing the thermal measurement process along the X-direction and the Y-direction, the method further includes providing an anti-reflection coating 306 on the transducer layer 302, and bonding the transducer layer 302 along with the anti-reflection coating 306 to the top surface S1 of the sample SX through the temporary bonding layer 304. In other words, the thermal measurement process is performed with the transducer layer 302 and the anti-reflection coating 306 covering the sample SX during measurement. For example, the pump laser 106A is configured to heat the designated region or designated spot 201 (shown in FIG. 1B) of the sample SX that is covered with the anti-reflection coating 306 and the transducer layer 302. In some embodiments, the anti-reflection coating 306 is provided on a support or a substrate (not shown), and bonded onto the transducer layer 302 along with the substrate. In certain embodiments, the substrate may be removed from the anti-reflection coating 306 during the thermal measurement process.

In the exemplary embodiment, the anti-reflection coating 306 includes a dielectric layer pair composed of a high refractive index material and a low refractive index material, wherein the high refractive index material is selected from the group consisting of titanium oxide (TiO₂), tantalum oxide (Ta₂O₃) and zirconium oxide (ZrO₂), and the low refractive index material is selected from the group consisting of silicon oxide (SiO₂) and magnesium fluoride (MgF₂). By using an anti-reflection coating 306 coated on the surface of the transducer layer 302 during the thermal measurement process, the heat absorption of the transducer layer 302 may be further improved, while a high reflectance can be maintained. As such, a 3D thermal imaging method with higher resolution can be achieved. In some embodiments, after performing the thermal measurement process as shown in FIG. 4B, the temporary bonding layer 304 along with the transducer layer 302 and the anti-reflection coating 306 are removed accordingly (e.g. through light or heat). As such, the damage to the sample SX can be minimized.

In the above embodiment, the transducer layer 302 and the anti-reflection coating 306 are bonded to the entire top surface S1 of the sample SX through the temporary bonding layer 304. However, the disclosure is not limited thereto. For example, in one embodiment shown in FIG. 5, the anti-reflection coating 306, the transducer layer 302 and the temporary bonding layer 304 are arranged as a filter FT on the objective lens 304, which may be repetitively used in the thermal measurement process at various spots along the X-Y direction of the sample SX. For example, the pump laser 106A may be used for emitting a first laser beam 108A that passes through the filter FT for heating a first designated spot of the sample SX, and the probe laser 106B may be used for emitting a second laser beam that passes through the filter FT to focus onto the first designated spot of the sample SX, and for detecting a thermal reflectance change of the sample SX. Thereafter, a second thermal measurement process may be performed on a second designated spot (or other spots along the X-Y direction) of the sample SX using the same filter FT. By using a filter FT including the anti-reflection coating 306, the transducer layer 302 and the temporary bonding layer 304 arranged on the objective lens 304 of the thermal measurement system 100, the heat absorption of the transducer layer 302 may be improved, a high reflectance can be maintained, and the thermal property measurement method is non-destructive. As such, a 3D thermal imaging method with higher resolution can be achieved.

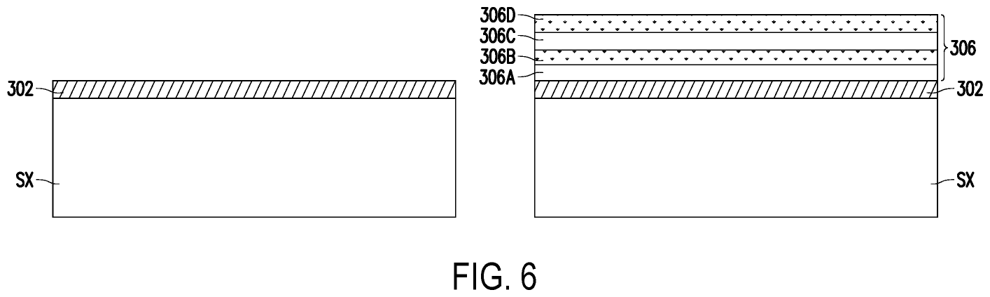

FIG. 6 is an example of comparing a thermal measurement process of a first sample coated with a transducer layer 302, and a second sample coated with a transducer layer 302 and an anti-reflection coating 306 located on top of the transducer layer 302. In the illustrated embodiment, in the first sample, an aluminum layer is used as a transducer layer 302 and bonded onto the top surface of a silicon substrate (the sample SX). Furthermore, in the second sample, besides having the aluminum layer (transducer layer 302) and the silicon substrate (the sample SX), an anti-reflection coating 306 having dielectric layer pairs composed of a high refractive index material layers 306A, 306C and a low refractive index material layers 306B, 306D are further coated on the transducer layer 302. In the exemplary embodiment, the high refractive index material layers 306A, 306C are made of titanium oxide (TiO₂), while the low refractive index material layers 306B, 306D are made of silicon oxide (SiO₂). Evaluation was conducted at a pump wavelength of 532 nm, and results indicated that the absorption is enhanced from approximately 7.8% to 68.8% (8 times enhanced) when the anti-reflection coating 306 is further included. Additionally, the reflectance remains high above 87% for both samples. These evaluation results indicated that when an anti-reflection coating 306 is further provided, the heat absorption of the transducer layer 302 may be further improved, while a high reflectance can be maintained. Overall, a 3D thermal imaging method with higher resolution can be achieved when the anti-reflection 306 is used.

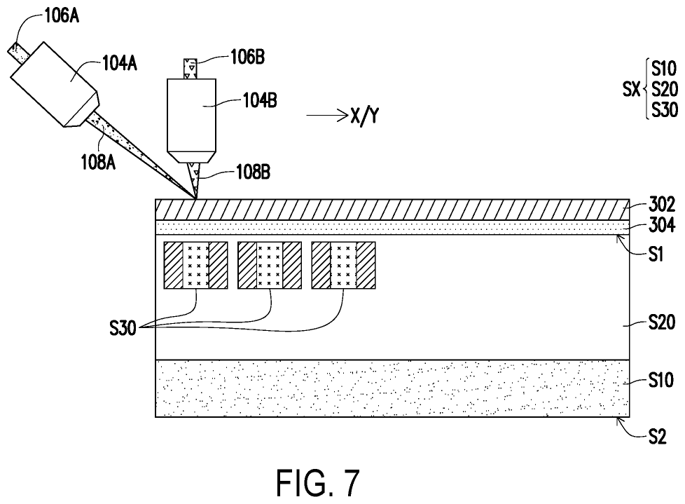

In the previous embodiments, the pump laser 106A and the probe laser 106B are focused using the same objective lens 104. However, the disclosure is not limited thereto. For example, referring to FIG. 7, the pump laser 106A and the probe laser 106 in the thermal measurement system 100 are focused using separated objective lens 104A, 104B. In some embodiments, during the thermal measurement process along the X-direction and the Y-direction, an angle of incidence of the pump laser 106A is non-collinear to an angle of incidence of the probe laser 106B. In other words, the first laser beam 108A emitted by the pump laser 106A is sent through the objective lens 104A to focus on the transducer layer 302, while the second laser beam 108B emitted by the probe laser 106B is sent through another objective lens 104B to focus on the transducer layer 302. In the exemplary embodiment, the objective lens 104A is arranged at an angle relative to the objective lens 104B so that the angle of incidence of the pump laser 106A is different from the angle of incidence of the probe laser 106B. In certain embodiments, the angle of incidence of the pump laser 106A is at the Brewster's angle for further improving the heat absorption of the transducer layer 302, while the angle of incidence of the probe laser 106B is normal to the surface of the transducer layer 302. The Brewster's angle will be different depending on the type of material used as the transducer layer 302. For example, in one embodiment, when the transducer layer 302 is aluminum, then the angle of incidence of the pump laser 106A at the Brewster's angle will be approximately 80° for a wavelength of 532 nm of the pump laser 106A. By using a non-collinear pump laser 106A for emitting the first laser beam 108A at the Brewster's angle, the heat absorption of the transducer layer 302 may be further improved. Overall, a 3D thermal imaging method with higher resolution can be achieved.

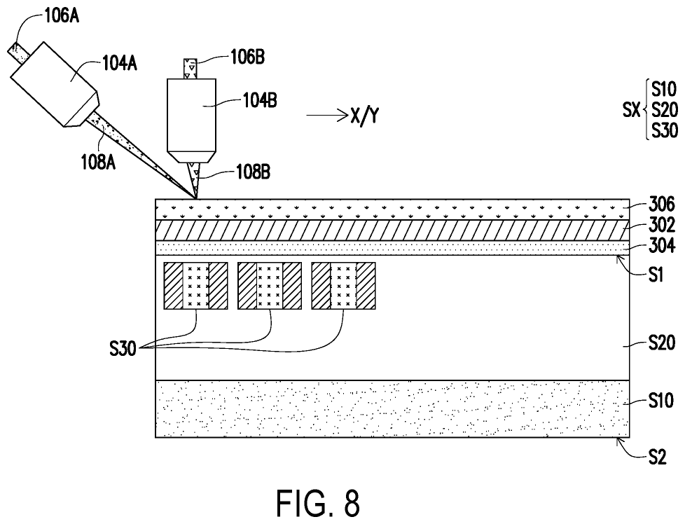

The embodiment illustrated in FIG. 8 is similar to the embodiment illustrated in FIG. 7, therefore the same reference numerals are used to refer to the same or liked parts, and its detailed description will not be repeated herein. The difference between the embodiments is that an anti-reflection coating 306 is further provided on the transducer layer 302 in FIG. 8. As illustrated in FIG. 8, in some embodiments, during the thermal measurement process along the X-direction and the Y-direction (or the X-Y plane), the angle of incidence of the pump laser 106A is different from the angle of incidence of the probe laser 106B, whereby the angle of incidence of the pump laser 106A is at the Brewster's angle for further improving the heat absorption of the transducer layer 302. By using a non-collinear pump laser 106A for emitting the first laser beam 108A at the Brewster's angle, and when an anti-reflection 306 coating is further provided, the heat absorption of the transducer layer 302 may be further improved, while a high reflectance can be maintained. Overall, a 3D thermal imaging method with higher resolution can be achieved.

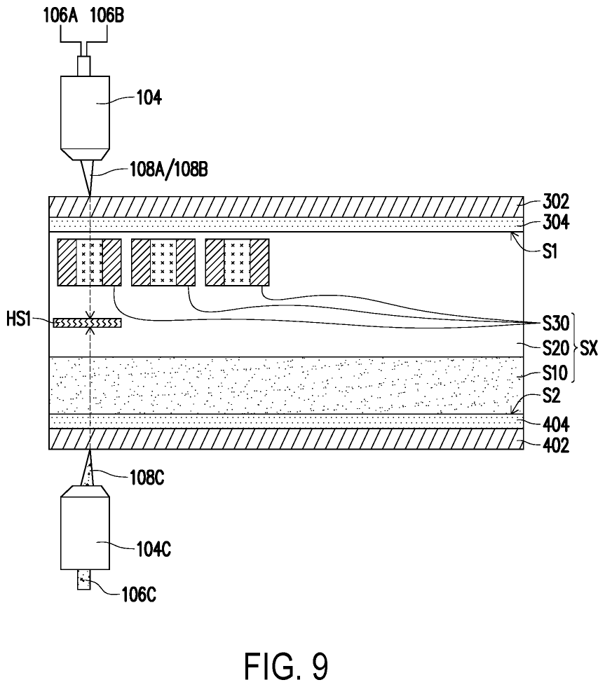

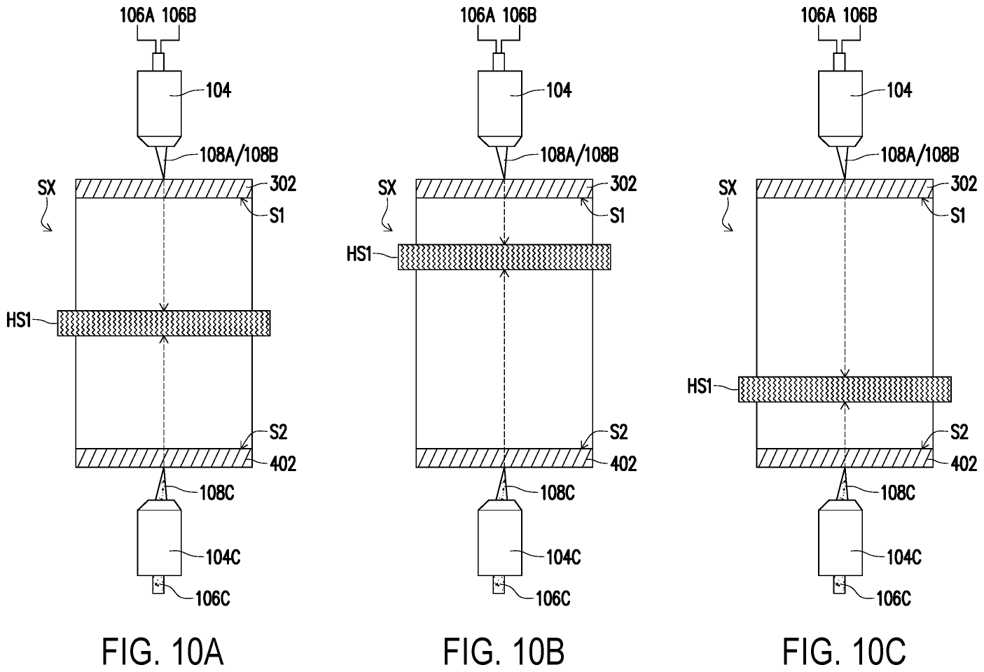

FIG. 9 to FIG. 10C are schematic sectional views of various steps in performing thermal measurement processes along a X-Z plane or a Y-Z plane in the thermal property measurement method in accordance with various embodiments of the present disclosure. Referring to FIG. 9, in some embodiments, the thermal measurement system 100 may further include an auxiliary pump laser 106C that is configured to emit a third laser beam 108C that is sent through another objective lens 104C to focus onto the sample SX. In the exemplary embodiment, a transducer layer 302 is placed on the top surface S1 of the sample SX through a temporary bonding layer 304, while another transducer layer 402 is placed on the bottom surface S2 of the sample SX through another temporary bonding layer 404. The transducer layer 302 and the transducer layer 402 may respectively be an aluminum layer, a gold layer, a copper layer, a plutonium layer, or a bi-layer consisting of aluminum and gold. Furthermore, a material of the transducer layer 302 may be the same as a material of the transducer layer 402, or may be different to a material of the transducer layer 402. In some embodiments, the transducer layer 402 is directly disposed on the sample stage 102 having an opening pattern, or on a substrate that is made of transparent glass, which allows the third laser beam 108C emitted by the auxiliary pump laser 106C to pass through.

In the illustrated embodiment, when performing the thermal measurement process along the Z-direction (along the X-Z plane or the Y-Z plane), the method includes using the pump laser 106A to heat a designated spot 201 (shown in FIG. 1B) of the sample SX from the top surface S1 of the sample SX, and using the auxiliary pump laser 106C to heat the designated spot 201 of the sample SX from a bottom surface S2 of the sample SX. Additionally, the method further includes using the probe laser 106B to focus on a center hot spot zone HS1 of the sample SX along the Z-direction for generating the reflectance signal from the designated spot 201 of the sample SX, and determining the thermal reflectance change over time from the reflectance signal. In the exemplary embodiment, the probe laser 106B is an infrared probe laser in order to see through the transducer layer 302 and for focusing inside the sample SX. In some embodiments, a pump frequency of the pump laser 106A and the auxiliary pump laser 106C are adjusted for adjusting the center hot spot zone HS1 of the sample SX along the Z-direction. Details of adjusting the center hot spot zone HS1 is described with reference to FIG. 10A to FIG. 10C.

Referring to FIG. 10A, in some embodiments, the center hot spot zone HS1 is at a center of the sample SX. In such embodiment, a heat penetration depth (Lp) to the center hot spot zone HS1 is controlled by adjusting the laser focus spot (FS) size in diameter of the pump laser 106A and the auxiliary pump laser 106C. For example, for a sample SX having a 20 μm thickness, the center of the sample SX is at the 10 μm thickness position. To adjust the center hot spot zone HS1 to such position, a top focus spot size (top FS) of the pump laser 106A is adjusted to be equal to a bottom focus spot size (bottom FS) of the auxiliary pump laser 106B, so that the top FS and the bottom FS are both 10 μm, and whereby the center hot spot zone HS1 or heat penetration depth (Lp) will be approximately located at the 10 μm thickness position. Furthermore, in some embodiments, the center hot spot zone HS1 or the heat penetration depth (Lp) can be controlled by the following formula:

$$L p = ( \sqrt{\frac{k}{C π f}} α \sqrt{\frac{1}{f}}$$

wherein, in the above formula, κ is the thermal conductivity, C is the heat capacity per unit volume, and f is the pump modulation frequency.

In other words, for controlling the center hot spot zone HS1 or heat penetration depth (Lp) to the 10 μm thickness position, the pump modulation frequency f is adjusted so that the heat penetration depth (Lp) in the formula above is equal to 10 μm. As such, the heating position along the Z direction may be adjusted based on the pump modulation frequency.

Referring to FIG. 10B, in some embodiments, the center hot spot zone HS1 is near the top surface S1 of the sample SX. For example, for a sample SX having a 20 μm thickness, the center hot spot zone HS1 may be adjusted to be at a 5 μm thickness position from the top surface S1. To adjust the center hot spot zone HS1 to such position, a top focus spot size (top FS) of the pump laser 106A is adjusted to be 5 μm, while the bottom focus spot size (bottom FS) of the auxiliary pump laser 106B is adjusted to be 15 μm. As such, the center hot spot zone HS1 or heat penetration depth (Lp) will be approximately located at the 5 μm thickness position from the top surface S1. Furthermore, the center hot spot zone HS1 or heat penetration depth (Lp) can also be controlled to the 5 μm thickness position from the top surface S1, which is accomplished by adjusting the pump modulation frequency f so that the heat penetration depth (Lp) in the formula above is equal to 5 μm.

Referring to FIG. 10C, in some embodiments, the center hot spot zone HS1 is near the bottom surface S2 of the sample SX. For example, for a sample SX having a 20 μm thickness, the center hot spot zone HS1 may be adjusted to be at a 15 μm thickness position from the top surface S1. To adjust the center hot spot zone HS1 to such position, a top focus spot size (top FS) of the pump laser 106A is adjusted to be 15 μm, while the bottom focus spot size (bottom FS) of the auxiliary pump laser 106B is adjusted to be 5 μm. As such, the center hot spot zone HS1 or heat penetration depth (Lp) will be approximately located at the 15 μm thickness position from the top surface S1. Furthermore, the center hot spot zone HS1 or heat penetration depth (Lp) can also be controlled to the 15 μm thickness position from the top surface S1, which is accomplished by adjusting the pump modulation frequency f so that the heat penetration depth (Lp) in the formula above is equal to 15 μm. In other words, a pump frequency f may be changed to adjust the center hot spot zone HS1 of the sample SX along the Z-direction when measuring a different spot/region along the Z-direction of the sample SX.

Based on the embodiments shown in FIG. 2 to FIG. 10C above, the thermal measurement processes along the X-direction, the Y-direction and the Z-direction can be performed to measure the thermal property or thermal conductivity of the sample SX, and for generating a three-dimensional thermal image of the sample SX. For example, a first designated spot 201 (shown in FIG. 1B) of the sample Sx may be heated using the pump laser 106A (and/or the auxiliary pump laser 106C) in the presence of the transducer layer 302 and/or the anti-reflection coating 306 based on the different embodiments above. Subsequently, the probe laser 106B may be used for generating a reflectance signal of the sample SX, and for determining a first thermal conductivity value in the designated spot 201 of the sample SX from the reflectance signal.

Furthermore, a second designated spot of the sample Sx (any spot in the sample SX) may be heated using the pump laser 106A (and/or the auxiliary pump laser 106C) in the presence of the transducer layer 302 and/or the anti-reflection coating 306 based on the different embodiments above. Subsequently, the probe laser 106B may be used for generating a reflectance signal of the sample SX, and for determining a second thermal conductivity value in the second designated spot of the sample SX from the reflectance signal. By conducting the thermal measurement processes at various spots of the sample SX, an effective thermal conductivity (κeff) of the sample SX based on the obtained thermal conductivity values (first, second thermal conductivity values etc.) at the various spots may be calculated. For example, the effective thermal conductivity (κeff) may be determined by the following formula:

$$keff = \sqrt{{k}_{⊥} {k}_{//}}$$

wherein, in the formula above, κ⊥ is the out-of-plane thermal conductivity, while κ// is the in-plane thermal conductivity.

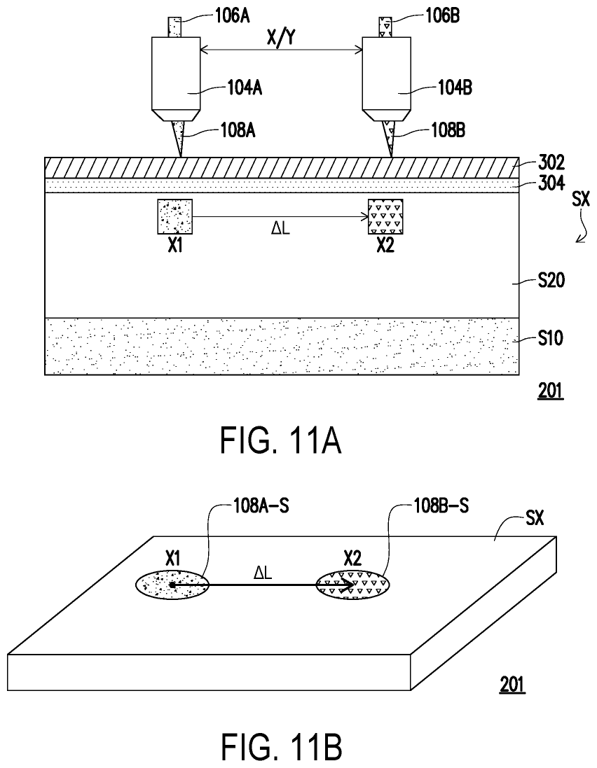

In FIG. 2 to FIG. 10C above, the thermal measurement processes are conducted at a single site at various spots to obtain a 3D mapping of thermal conductivity of the sample SX. In some embodiments, the thermal measurement processes described in FIG. 11A to FIG. 15B may also be performed to obtain the in-plane and out-of-plane thermal conductivity, and for evaluating the directional temperature change or heat dissipation characteristics of the sample SX.

FIG. 11A to FIG. 13B are schematic sectional and three-dimensional views of various steps in performing thermal measurement processes along a X-Y plane in the thermal property measurement method in accordance with some other embodiments of the present disclosure. FIG. 11A and FIG. 11B shows an enlarged view of a designated spot 201 or designated region 201 of the sample SX. As illustrated in FIG. 11A and FIG. 11B, the first laser beam 108A emitted by the pump laser 106A is sent through the objective lens 104A to focus on the transducer layer 302, while the second laser beam 108B emitted by the probe laser 106B is sent through another objective lens 104B to focus on the transducer layer 302.

In some embodiments, when performing the thermal measurement processes along the X-Y plane, the method includes heating a first site X1 in the designated region 201 of the sample SX covered with the transducer layer 302 using the pump laser 106A. Additionally, the method further includes using the probe laser 106B to scan from a center of the first site X1 (the hottest spot) to a second site X2 (the coldest spot) in the designated region 201 for generating the reflectance signal, and so that the probe laser 106B is offset from the pump laser 106A by a distance of ΔL. In the exemplary embodiment, a focus spot size 108A-S of the first laser beam 108A emitted by the pump laser 106A is equal to a focus spot size 108B-S of the second laser beam 108B emitted by the probe laser 106B. The offset distance ΔL of the probe laser 106B from the pump laser 106A may be adjusted to determine a temperature change or heat dissipation characteristics from the first site X1 to the second site X2. Furthermore, the offset distance ΔL is in a range of 10 μm to 0.1 mm.

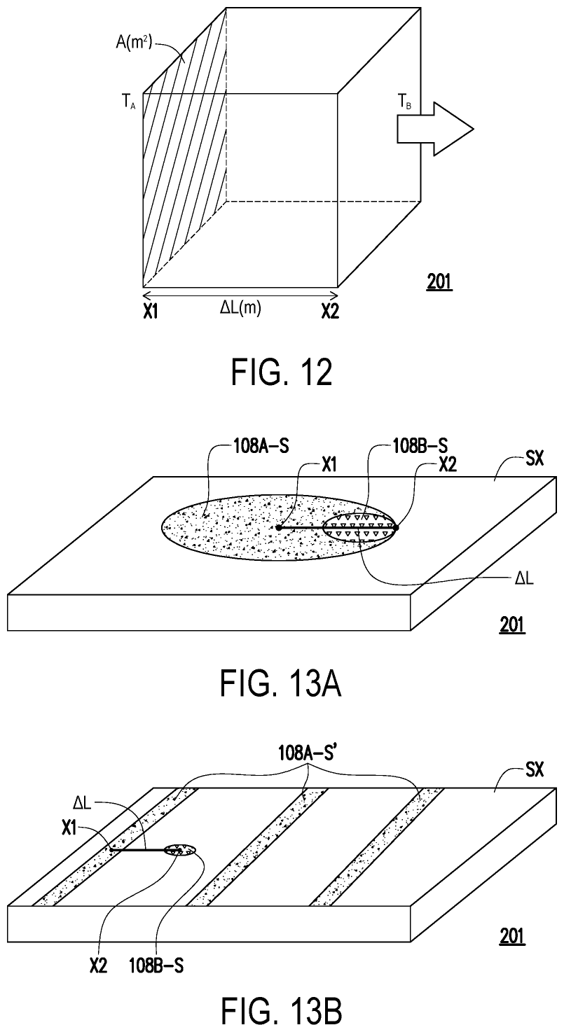

In the exemplary embodiment, a magnitude of the reflectance signal generated is a function of a temperature of the sample SX, whereby the in-plane thermal conductivity (κ//) along the X-Y plane in the designated region 201 of the sample SX is determined by using Fourier's law of heat conduction as shown in FIG. 12, which is represented by the following formula:

$${κ}_{//} = Q Δ L / ( {T}_{A} - {T}_{B} ) A$$

wherein, as shown in FIG. 12 and in the formula above, Q is an amount of heat flow based on the pump laser power; the distance ΔL is the offset distance of the probe laser 106B from the pump laser 106A; T_{A} is a temperature determined by the probe laser 106B at the first site X1; T_{B} is a temperature determined by the probe laser 106B at the second site X2; and A is heat transfer cross-sectional area of the designate region 201 of the sample SX.

By using the method illustrated in FIG. 11A to FIG. 11B, the directional in-plane thermal conductivity along the X-Y plane can be determined with improved resolution, and thermal responses such as heat dissipation in a specific direction (e.g. from first site X1 to second site X2) can be monitored and evaluated.

FIG. 13A illustrates an alternative method of operating the pump laser 106A and the probe laser 106B as shown in FIG. 11A. Referring to FIG. 13A, in some embodiments, when performing the thermal measurement processes along the X-Y plane, the method includes heating a first site X1 in the designated region 201 of the sample SX covered with the transducer layer 302 using the pump laser 106A. Additionally, the method further includes using the probe laser 106B to scan from a center of the first site X1 to a second site X2 located at a periphery of the first site X1 in the designated region 201 for generating the reflectance signal, and so that the probe laser 106B is offset from the pump laser 106A by a distance of ΔL. In the exemplary embodiment, a focus spot size 108A-S of the pump laser 106A is greater than a focus spot size 108B-S of the probe laser 106B. Furthermore, the offset distance ΔL is in a range of 1 μm to 10 μm. In the exemplary embodiment, the in-plane thermal conductivity (κ//) along the X-Y plane in the designated region 201 of the sample SX is determined by using Fourier's law of heat conduction as shown in FIG. 12, and determined by the same formula shown above. By using the method illustrated in FIG. 13A, the directional in-plane thermal conductivity along the X-Y can be determined with further improved spatial resolution (<10 μm), and thermal responses such as heat dissipation in a specific direction (e.g. from first site X1 to second site X2) can be monitored and evaluated at higher precision.

FIG. 13B illustrates an alternative method of operating the pump laser 106A and the probe laser 106B as shown in FIG. 11A. Referring to FIG. 13B, in some embodiments, the pump laser 106A is configured to emit a first laser beam 108A through a photomask pattern, so that the emitted first laser beam 108A has grating patterns 108A-S′. In the exemplary embodiment, when performing the thermal measurement processes along the X-Y plane, the method includes heating a first site X1 in the designated region 201 of the sample SX covered with the transducer layer 302 using the pump laser 106A with grating patterns 108A-S′. Additionally, the method further includes using the probe laser 106B to scan from a center of the first site X1 (hottest spot) to a second site X2 (coldest spot) that is located at a center position in between two adjacent grating patterns 108A-S′ in the designated region 201. Thereafter, the reflectance signal can be generated, and wherein the probe laser 106B is offset from the pump laser 106A by a distance of ΔL, and the offset distance ΔL is less than 1 μm. In the exemplary embodiment, the in-plane thermal conductivity (κ//) along the X-Y plane in the designated region 201 of the sample SX is determined by using Fourier's law of heat conduction as shown in FIG. 12, and determined by the same formula shown above. By using the method illustrated in FIG. 13B, the directional in-plane thermal conductivity along the X-Y can be determined with further improved spatial resolution (<1 μm), and thermal responses such as heat dissipation in a specific direction (e.g. from first site X1 to second site X2) can be monitored and evaluated at higher precision.

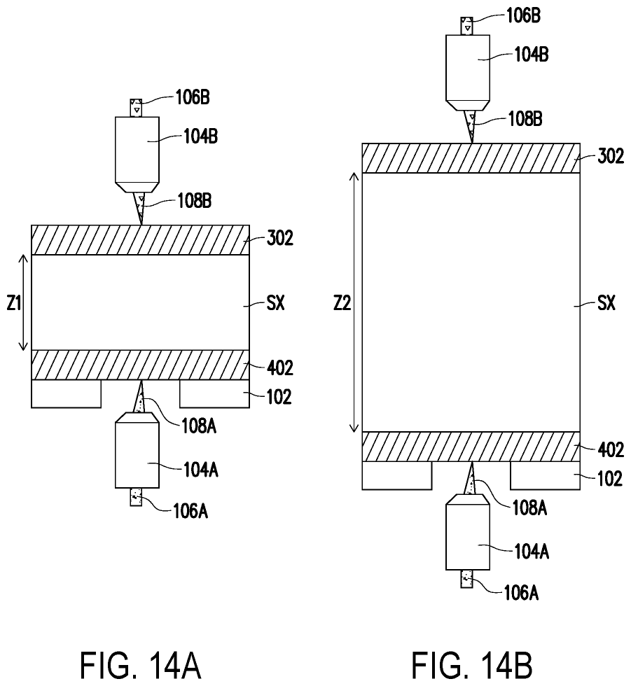

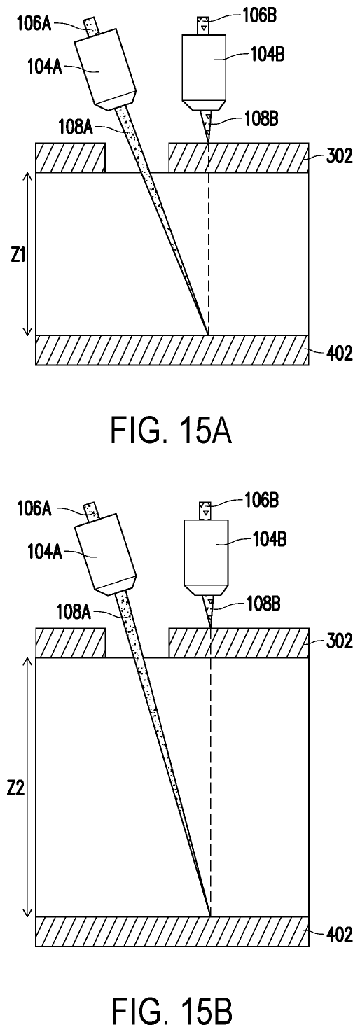

FIG. 14A to FIG. 15B are schematic sectional views of various steps in performing thermal measurement processes along a X-Z plane or a Y-Z plane in the thermal property measurement method in accordance with some other embodiments of the present disclosure. Referring to FIG. 14A to FIG. 14B, multiple samples SX with different thicknesses are provided, whereby each of the samples SX are provided with transducer layers (302, 402) on top and bottom surfaces. In the exemplary embodiment, a first sample SX with a thickness of Z1 and a second sample SX with a thickness of Z2 is provided. The transducer layers 402 are directly disposed on the sample stage 102 (or sample holder) having an opening pattern, or on a substrate that is made of transparent glass, which allows the first laser beam 108A emitted by the pump laser 106A to pass through.

In some embodiments, when performing the thermal measurement processes along a Z-direction (the X-Z plane or Y-Z plane), the method includes using the pump laser 106A to emit a first laser beam 108A that is sent through the objective lens 104A to heat a bottom surface of the first sample SX having the thickness of Z1. The method further includes using the probe laser 106B to emit a second laser beam 108B that is sent through the objective lens 104B to focus onto a top surface of the first sample SX having the thickness of Z1, and for detecting a thermal reflectance change of the sample SX.

In addition, the method further includes using the pump laser 106A to emit a first laser beam 108A that is sent through the objective lens 104A to heat a bottom surface of the second sample SX having the thickness of Z2. The method further includes using the probe laser 106B to emit a second laser beam 108B that is sent through the objective lens 104B to focus onto a top surface of the second sample SX having the thickness of Z2, and for detecting a thermal reflectance change of the sample SX.

In the exemplary embodiment, a magnitude of the reflectance signal generated is a function of a temperature of the sample SX, whereby the out-of-plane thermal conductivity (κ⊥) along the X-Z plane and the Y-Z in the designated region 201 of the sample SX is determined by using Fourier's law of heat conduction similar to that shown in FIG. 12, and is represented by the following formula:

$$κ ⊥ = Q Δ Z / ( {T}_{A} - {T}_{B} ) A$$

wherein, as shown in FIG. 12 and in the formula above, Q is an amount of heat flow based on the pump laser power; ΔZ is the difference (Z1-Z2) between the thicknesses of the measured samples SX, T_{A} is a temperature determined by the probe laser 106B from the thermal reflectance changes obtained from the first sample (thickness Z1); T_{B} is a temperature determined by the probe laser 106B from the thermal reflectance changes obtained from the second sample (thickness Z2); and A is a heat transfer cross-sectional area, which is based on the pump/probe focus spot size (same for both samples).

As such, by using the method illustrated in FIG. 14A to FIG. 14B, the directional out-of-plane thermal conductivity along the X-Z and Y-Z plane can be determined with improved resolution, and thermal responses such as heat dissipation in a specific direction (in thickness range of ΔZ) can be monitored and evaluated.

The embodiment shown in FIG. 15A to FIG. 15B is similar to the embodiment shown in FIG. 14A to FIG. 14B. Therefore, the same reference numerals are used to refer to the same or liked parts, and its detailed description will not be repeated herein. Referring to FIG. 15A and FIG. 15B, multiple samples SX with different thicknesses are provided, whereby each of the samples SX are provided with transducer layers (302, 402) on top and bottom surfaces. In the exemplary embodiment, a first sample SX with a thickness of Z1 and a second sample SX with a thickness of Z2 is provided. The transducer layers 302 on the top surface of the samples SX has an opening pattern that allows the first laser beam 108A emitted by the pump laser 106A to pass through.

In some embodiments, when performing the thermal measurement processes along a Z-direction (the X-Z plane or Y-Z plane), the method includes using the pump laser 106A to emit a first laser beam 108A that is sent through the objective lens 104A, and sent through a top surface of the first sample SX to heat a bottom surface of the first sample SX having the thickness of Z1. In some embodiments, the angle of incidence of the pump laser 106A is at the Brewster's angle for further improving the heat absorption of the transducer layer 402. In some embodiments, the method further includes using the probe laser 106B that is non-collinear to the pump laser 106A, to emit a second laser beam 108B that is sent through the objective lens 104B to focus onto a top surface of the first sample SX having the thickness of Z1, and for detecting a thermal reflectance change of the sample SX.

In addition, the method further includes using the pump laser 106A to emit a first laser beam 108A that is sent through the objective lens 104A, and sent through a top surface of the second sample SX to heat a bottom surface of the second sample SX having the thickness of Z2. In some embodiments, the angle of incidence of the pump laser 106A is at the Brewster's angle for further improving the heat absorption of the transducer layer 402. In some embodiments, the method further includes using the probe laser 106B that is non-collinear to the pump laser 106A, to emit a second laser beam 108B that is sent through the objective lens 104B to focus onto a top surface of the second sample SX having the thickness of Z2, and for detecting a thermal reflectance change of the sample SX.

In the exemplary embodiment, the out-of-plane thermal conductivity (κ⊥) along the X-Z plane and the Y-Z in the designated region 201 of the sample SX is determined by using Fourier's law of heat conduction similar to that shown in FIG. 12, and determined by using the same formula described for FIG. 14A and FIG. 14B above. As such, by using the method illustrated in FIG. 15A to FIG. 15B, the directional out-of-plane thermal conductivity along the X-Z and Y-Z plane can be determined with improved resolution, and thermal responses such as heat dissipation in a specific direction (in thickness range of ΔZ) can be monitored and evaluated.

According to the above embodiments, in the thermal property measurement method of the present disclosure, a transducer layer is bonded on a sample through a temporary bonding layer prior to a thermal property measurement process, and thermal measurement processes along a X-Y plane, a X-Z plane and Y-Z plane of the sample are performed to generate a three-dimensional thermal image of the sample by mapping the temperature change and the thermal conductivity of the sample. As such, a 3D thermal imaging method that is non-destructive with higher resolution can be achieved, which will meet the demands in characterizing 3D heat dissipation in developing transistors and chip bonding structures, or directional heat transport design structures in the back-end-of Line (BEOL) of fabrication.

In accordance with some embodiments of the present disclosure, a thermal property measurement method includes the following steps. A thermal measurement system is provided. The thermal measurement system includes a sample stage, a pump laser and a probe laser. A sample is provided on the sample stage, and a thermal measurement process is performed by the following steps. A designated spot of the sample is heated using the pump laser. The probe laser is used for generating a reflectance signal from the designated spot of the sample, and a thermal reflectance change over time is determined from the reflectance signal. A temperature change and a thermal conductivity of the sample at the designated spot is calculated from the thermal reflectance change. The thermal measurement process is repeated to obtain the temperature change and the thermal conductivity of the sample at various spots along a X-direction of the sample. The thermal measurement process is repeated to obtain the temperature change and the thermal conductivity of the sample at various spots along a Y-direction of the sample, wherein the Y-direction is perpendicular to the X-direction. The thermal measurement process is repeated to obtain the temperature change and the thermal conductivity of the sample at various spots along a Z-direction of the sample, wherein the Z-direction is perpendicular to the X-direction and the Y-direction. A three-dimensional thermal image of the sample is generated by mapping the temperature change and the thermal conductivity of the sample obtained at the various spots along the X-direction, the Y-direction and the Z-direction.

In accordance with some other embodiments of the present disclosure, a thermal property measurement method includes the following steps. A sample for thermal property measurement is provided. A transducer layer is bonded on the sample through a temporary bonding layer. A three-dimensional measurement process of the sample is performed by performing thermal measurement processes along a X-Y plane, a X-Z plane and Y-Z plane of the sample, wherein the X-Y plane is parallel to a top surface of the sample, and the X-Z plane and Y-Z plane are perpendicular to the top surface of the sample. Each thermal measurement processes include heating a designated region of the sample covered with the transducer layer using a pump laser, and using a probe laser for generating a reflectance signal of the sample, and determining a thermal conductivity in the designated region of the sample from the reflectance signal. Furthermore, the transducer layer is removed along with the temporary bonding layer from the top surface of the sample.

In accordance with yet another embodiment of the present disclosure, a method of measuring thermal conductivity includes the following steps. A sample for thermal conductivity measurement is provided. A transducer layer is bonded on the sample. A designated spot of the sample covered with the transducer layer is heated using a pump laser. A probe laser is used for generating a reflectance signal of the sample, and a first thermal conductivity value in the designated spot of the sample is determined from the reflectance signal, wherein an angle of incidence of the pump laser is non-collinear to an angle of incidence of the probe laser.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure.

What is claimed is:

1. A thermal property measurement method, comprising:
providing a thermal measurement system, comprising a sample stage, a pump laser and a probe laser;
providing a sample on the sample stage, and performing a thermal measurement process by:
heating a designated spot of the sample using the pump laser;
using the probe laser for generating a reflectance signal from the designated spot of the sample, and determining a thermal reflectance change over time from the reflectance signal; and
calculating a temperature change and a thermal conductivity of the sample at the designated spot from the thermal reflectance change;
repeating the thermal measurement process to obtain the temperature change and the thermal conductivity of the sample at various spots along a X-direction of the sample;
repeating the thermal measurement process to obtain the temperature change and the thermal conductivity of the sample at various spots along a Y-direction of the sample, wherein the Y-direction is perpendicular to the X-direction;
repeating the thermal measurement process to obtain the temperature change and the thermal conductivity of the sample at various spots along a Z-direction of the sample, wherein the Z-direction is perpendicular to the X-direction and the Y-direction; and
generating a three-dimensional thermal image of the sample by mapping the temperature change and the thermal conductivity of the sample obtained at the various spots along the X-direction, the Y-direction and the Z-direction.

2. The method according to claim 1, wherein after providing the sample on the sample stage, and prior to performing the thermal measurement process along the X-direction and the Y-direction, the method further comprises:
providing a transducer layer on a top surface of the sample;
providing an anti-reflection coating on the transducer layer; and
performing the thermal measurement process with the transducer layer and the anti-reflection coating covering the sample during measurement.

3. The method according to claim 2 wherein the method further comprises:
bonding the transducer layer to the top surface of the sample through a temporary bonding layer; and
after performing the thermal measurement process, removing the temporary bonding layer along with the transducer layer from the top surface of the sample.

4. The method according to claim 1, wherein the pump laser and the probe laser in the thermal measurement system are focused using separated objective lens, and during the thermal measurement process along the X-direction and the Y-direction, an angle of incidence of the pump laser is non-collinear to an angle of incidence of the probe laser.

5. The method according to claim 4, wherein the angle of incidence of the pump laser is at the Brewster's angle.

6. The method according to claim 1, wherein the thermal measurement system further comprises an auxiliary pump laser, and when performing the thermal measurement process along the Z-direction, the method further comprises:
using the pump laser to heat the designated spot of the sample from a top surface of the sample;
using the auxiliary pump laser to heat the designated spot of the sample from a bottom surface of the sample;
using the probe laser to focus on a center hot spot zone of the sample along the Z-direction for generating the reflectance signal from the designated spot of the sample, and determining the thermal reflectance change over time from the reflectance signal; and
changing a pump frequency to adjust the center hot spot zone of the sample along the Z-direction when measuring a different spot in the various spots along the Z-direction of the sample.

7. The method according to claim 6, wherein the probe laser is an infrared probe laser.

8. The method according to claim 1, wherein the sample is a semiconductor device including transistors.

9. A thermal property measurement method, comprising:
providing a sample for thermal property measurement;
bonding a transducer layer on the sample through a temporary bonding layer;
performing a three-dimensional measurement process of the sample by performing a plurality of thermal measurement processes along a X-Y plane, a X-Z plane and Y-Z plane of the sample, wherein the X-Y plane is parallel to a top surface of the sample, and the X-Z plane and Y-Z plane are perpendicular to the top surface of the sample, and each of the plurality of thermal measurement processes comprises:
heating a designated region of the sample covered with the transducer layer using a pump laser, and using a probe laser for generating a reflectance signal of the sample, and determining a thermal conductivity in the designated region of the sample from the reflectance signal; and
removing the transducer layer along with the temporary bonding layer from the top surface of the sample.

10. The method according to claim 9, further comprising:
providing an anti-reflection coating on the transducer layer prior to performing the three-dimensional measurement process; and
heating the designated region of the sample covered with the anti-reflection coating and the transducer layer using the pump laser.

11. The method according to claim 10, wherein the anti-reflection coating comprises a dielectric layer pair composed of a high refractive index material and a low refractive index material, wherein the high refractive index material is selected from the group consisting of titanium oxide (TiO₂), tantalum oxide (Ta₂O₃) and zirconium oxide (ZrO₂), and the low refractive index material is selected from the group consisting of silicon oxide (SiO₂) and magnesium fluoride (MgF₂).

12. The method according to claim 9, wherein performing a plurality of thermal measurement processes along the X-Y plane comprises:
heating a first site in the designated region of the sample covered with the transducer layer using the pump laser;
using the probe laser to scan from a center of the first site to a second site in the designated region for generating the reflectance signal, and so that the probe laser is offset from the pump laser by a distance of ΔL; and
determining the thermal conductivity (κ//) in the designated region of the sample by the following formula:
$${κ}_{//} = Q Δ L / ( {T}_{A} - {T}_{B} ) A$$
wherein Q is an amount of heat flow based on the pump laser power;
the distance ΔL is the offset distance of the probe laser from the pump laser;
T_{A} is a temperature determined by the probe laser at the first site;
T_{B} is a temperature determined by the probe laser at the second site; and
A is heat transfer cross-sectional area of the designate region of the sample.

13. The method according to claim 12, wherein a focus spot size of the pump laser is greater than a focus spot size of the probe laser.

14. The method according to claim 9, wherein performing a plurality of thermal measurement processes along the X-Z plane and the Y-Z plane comprises:
heating a top surface of the designated region of the sample covered with the transducer layer using the pump laser;
heating a bottom surface of the designated region of the sample covered with another transducer laser using an auxiliary pump laser; and
using the probe laser to focus on a center hot spot zone of the sample along the X-Z plane or the Y-Z plane for generating the reflectance signal of the sample, and determining the thermal conductivity in the designated region of the sample from the reflectance signal.

15. The method according to claim 9, wherein during the plurality of thermal measurement processes, the pump laser and the probe laser are focused using separated objective lens, and wherein an angle of incidence of the pump laser is non-collinear to an angle of incidence of the probe laser.

16. The method according to claim 15, wherein the angle of incidence of the pump laser is at the Brewster's angle.

17. A method of measuring thermal conductivity, comprising:
providing a sample for thermal conductivity measurement;
bonding a transducer layer on the sample;
heating a designated spot of the sample covered with the transducer layer using a pump laser; and
using a probe laser for generating a reflectance signal of the sample, and determining a first thermal conductivity value in the designated spot of the sample from the reflectance signal, wherein an angle of incidence of the pump laser is non-collinear to an angle of incidence of the probe laser.

18. The method according to claim 17, wherein the transducer layer is an aluminum layer, a gold layer, a copper layer, a plutonium layer, or a bi-layer consisting of aluminum and gold.

19. The method according to claim 17, further comprising:
heating a second designated spot from a top surface of the sample covered with the transducer layer using the pump laser, and heating the second designated spot from a bottom surface of the sample covered with another transducer layer using an auxiliary pump laser; and
using the probe laser for generating a second reflectance signal of the sample, and determining a second thermal conductivity value in the second designated spot of the sample from the second reflectance signal; and
calculating an effective thermal conductivity of the sample based on at least the first thermal conductivity value and the second thermal conductivity value.

20. The method according to claim 17, wherein the transducer layer is bonded on the sample through a temporary bonding layer, and the temporary bonding layer includes a material selected from the group consisting of germanium (Ge), silicon germanium (Si₁₋ₓGeₓ), silicon carbide (SiC), phosphorus-doped silicon (Si:P), boron-doped silicon (Si:B), boron-doped silicon germanium (SiGe:B), phosphorus-doped silicon germanium (SiGe:P), yttrium oxide (Y₂O₃), cerium oxide (CeO₂), boron nitride (BN), gallium phosphide (GaP), and titanium nitride (TiN).
